(19) United States
(12) Patent Application Publication
(10) Pub. No.: US 2005/0021138 A1
(43) Pub. Date: Jan. 27, 2005

(54) Telescopic intraocular lens implant for treating age-related macular degeneration

(76) Inventor: Randall Woods, Prescott, AZ (US)

Correspondence Address: HOVEY WILLIAMS LLP, Suite 400, 2405 Grand, Kansas City, MO, 64108, US

(21) Appl. No.: 10/921,533

(22) Filed: Aug. 19, 2004

Related U.S. Application Data
(63) Continuation of application No. 10/280,956, filed on Oct. 25, 2002, now abandoned.

(51) Int. Cl. A61F 2/16

(52) U.S. Cl. 623/6.34; 623/6.37; 623/6.4

(57) ABSTRACT

An intraocular lens having an anterior lens member (48) presenting an anterior light-converging optic (52) and a posterior lens member (50) presenting a posterior light-diverging optic (68) for magnifying an observed image onto large regions of the retina (32) to permit central focus in patients suffering from AMD and a method of implanting the lens into the human eye (10). The anterior light-converging optic (52) is operably coupled with a flexible body (58) which extends radially therefrom and presents opposing bights (62) presenting termini (66) when the lens is viewed in cross-section. The posterior light-diverging optic (68) is operably coupled with an annular flange (76) which is arcuate in cross-section and mates with termini. Both the anterior and the posterior lens members (48, 50) have positioning holes (70, 80) formed therein permitting surgical implantation thereof. The IOL (46) is constructed of a flexible synthetic resin material such as polymethylmethacrylate and permits focusing upon objects located near to and far from the viewer.

### RELATED APPLICATIONS

This application is a continuation application of U.S. patent application Ser. No. 10/280,956, filed on Oct. 25, 2002, incorporated herein by reference.

### BACKGROUND OF THE INVENTION

1. Field of the Invention

The present invention relates to a telescopic intraocular lens implant (IOL) which replaces the natural crystalline lens for treating age-related macular degeneration (AMD). The IOL is inserted into the natural biological capsule and accommodates in response to the action of the ciliary body for focusing upon objects located near and far from the viewer.

2. Description of the Prior Art

AMD, the major cause of blindness in the western world, is caused by degeneration of the macular tissue of the retina responsible for sharp central vision. Two types of AMD are known. Wet or exudative AMD is caused by ingrowth blood vessels located in the choroid and is the most severe form of AMD. The swelling of these vessels and eventual leakage into the retina causes the destruction of macular tissue. The second type of AMD, dry or atropic AMD, is the most prevalent. The cause of dry AMD is not known, but is likely caused by a combination of thinning of the macular tissue and drusen deposit between the retinal pigmented epithelium (RPE) and Bruch's membrane. Drusen deposit is associated with dysfunctional cellular metabolism in the RPE. Both types of AMD cause severe disruption of vision acuity and afflict millions of individuals worldwide. Some forms of laser surgery may alleviate the rate of progression of wet AMD by destroying swollen blood vessels, however, no effective treatment for dry AMD is available.

Telescopic lenses for slightly improving visual acuity in patients suffering from AMD are known. However, these lenses are worn as external spectacle lenses and are much too heavy to be used properly. Recent efforts have been launched to develop IOLs for treating AMD comparable to those used to treat cataracts. These IOLs are ineffective because they are much too heavy and require a 13 mm incision of the cornea and biological capsule for implantation. The large incision size required to implant the heavy-weight lenses necessitates the use of sutures which may cause severe bleeding in the eye. Furthermore, the heavy weight of these IOLs can cause the cornea to reshape resulting in astigmatism. A great need exists for a lightweight telescopic IOL for treating AMD that can be embedded into the biological capsule without requiring an incision greater than 5 mm for implantation thereof. A small incision size is desirable because the eye can repair itself at incisions of about 1 to 5 mm and therefore does not require suturing. The risk of bleeding from sutures is therefore minimized.

Various IOLs have been used to treat cataracts. The first implant of an IOL within the eye to treat cataracts occurred in 1949. This experimental surgery attempted to place the replacement lens in the posterior chamber of the eye behind the iris. Problems such as dislocation after implantation forced abandonment of this approach, and for some period thereafter IOLs were implanted in the anterior chamber of the eye.

Others returned to the practice of inserting the IOL in the area of the eye posterior to the iris, known as the posterior chamber. This is the area where the patient's natural crystalline lens is located. When the IOL is located in this natural location, substantially normal vision may be restored to the patient and the problems of forward displacement of the vitreous humor and retina detachment encountered in anterior chamber IOLs are less likely to occur. IOLs implanted in the posterior chamber are disclosed in U.S. Pat. Nos. 3,718,870, 3,866,249, 3,913,148, 3,925,825, 4,014,049, 4,041,552, 4,053,953, and 4,285,072. None of these IOLs have accommodation capability.

IOLs capable of focusing offered the wearer the closest possible substitute to the crystalline lens. U.S. Pat. No. 4,254,509 to Tennant discloses an IOL which moves in an anterior direction upon contraction of the ciliary body and which is located anterior to the iris. Although the Tennant IOL possesses accommodation capabilities, it presents the same disadvantages as other anterior chamber lenses. U.S. Pat. No. 4,253,199 to Banko approaches the problem of providing a focusable IOL in a different manner, by providing a replacement IOL of deformable material sutured to the ciliary body. This IOL functions in much the same manner as the natural crystalline lens, but may cause bleeding because it requires sutures.

U.S. Pat. No. 4,409,691 to Levy is asserted to provide an accommodating IOL positioned within the capsule. This IOL is located in the posterior area of the capsule and is biased toward the fovea or rear of the eye. The Levy IOL is deficient because it requires the ciliary muscle to exert force through the zonules on the capsule in order to compress the haptics inward and drive the optic forward for near vision. However, the ciliary muscles do not exert any force during contraction because the zonules, being flexible filaments, exert only tension, not compression on the capsule. The natural elasticity of the IOL causes the capsule to become more spherical upon contraction of the ciliary muscle. Thus, there is no inward force exerted on the capsule to compress the haptics of the Levy IOL, and therefore accommodate for near vision. Even if such force were somehow available, the Levy IOL's haptics are loaded inward when accommodating for near vision. Since accommodation for near vision is the normal status of the capsule, the Levy IOL's haptics are loaded, reducing the fatigue life of the springlike haptics.

U.S. Pat. No. 5,674,282 to Cumming is directed towards an accommodating IOL for implanting within the capsule of an eye. The Cumming IOL comprises a central optic and two plate haptics which extend radially outward from diametrically opposite sides of the optic and are movable anteriorly and posteriorly relative to the optic. However, the Cumming IOL suffers from the same shortcomings as the Levy IOL in that the haptics are biased anteriorly by pressure from the ciliary bodies. This will eventually lead to pressure necrosis of the ciliary body.

Finally, U.S. Pat. No. 4,842,601 to Smith discloses an accommodating IOL having anterior and posterior members which urge against the anterior and posterior walls of the natural lens capsule. The muscular action exerted on the natural capsule will thus cause the IOL to flatten, thereby changing the focus thereof. The Smith IOL is formed of first and second plastic lens members connected to one another adjacent their peripheral edges so as to provide a cavity therebetween. The connection between the lens members is accomplished by way of a U-shaped flange on the first member which forms an inwardly facing groove for receiving an outwardly extended flange on the second member. The Smith IOL is faulty because the structure of the lens members makes surgical implantation thereof extremely difficult to accomplish, even for highly skilled surgeons. The Smith patent does not disclose converging and diverging optics, and also requires a large incision for placement of the IOL into the capsule of the bag.

The IOLs replaced the opaque crystalline lens symptomatic of cataracts through a small incision in the cornea and anterior wall of the biological capsule. The IOLs for the treatment of cataracts differed from the present invention in that the present invention utilizes diverging and converging lenses to magnify the image being viewed onto undamaged portions of the retina. Furthermore, the present invention can be utilized to treat both cataracts and AMD which can often occur simultaneously.

There is a long-felt need for a lightweight IOL capable of focusing in a manner similar to the natural lens for treating AMD and cataracts. This IOL should be readily insertable into the capsule and should last for a substantial number of years without damaging any of the eye components.

### SUMMARY OF THE INVENTION

The IOL of the present invention addresses this need because it provides a lightweight accommodating telescopic IOL for placement within the confines of the capsule of the human eye. The present invention presents a significant advance in the art because it provides a safe and efficient treatment for AMD.

The IOL of the present invention comprises an anterior and a posterior lens member operably coupled together to change shape in response to zonular movement. The anterior lens member includes an anterior optic that converges light. The light-converging anterior optic is operably coupled with a flexible body which extends radially and posteriority from the periphery of the anterior light-converging optic. In cross-section, the flexible body forms a pair of opposing bights presenting corresponding termini. The posterior lens member includes a light-diverging posterior optic which is operably coupled with a connector, preferably an annular flange that is arcuate in cross-section, that mates with the termini.

Implantation of the IOL of the present invention occurs in two segments. The posterior lens member is implanted within the capsule of the eye first, followed by the anterior lens member. The lens members are connected together to yield a unitary IOL capable of accommodation. Accommodation refers to the process by which the focal length of the IOL is changed in response to the contraction and relaxation of the ciliary body and is needed to permit focusing upon objects located far from and near to the viewer. The lens members and the optics are made of flexible synthetic resin material comprised of silicones, acrylates (such as polymethylmethacrylates), and mixtures thereof. Contraction of the ciliary body results in the relaxation of the zonular fibers thereby affecting the focal length of the lens. The IOL becomes more spheroid in shape and permits the viewer to focus upon objects located near to the viewer. When the object being viewed is located at a distance, the ciliary body relaxes and the zonular fibers contract causing the IOL to become discoid in shape.

Parallel rays of light are refracted through the light-converging anterior optic, and once converged, through the light-diverging posterior optic. The image being viewed is thus diverged onto a large region of the retina. This operation of the converging and diverging optics results in magnification of the image onto undamaged regions of the retina thus enabling improved vision.

### BRIEF DESCRIPTION OF THE DRAWINGS

FIG. 1 is a vertical sectional view showing an IOL of the invention within the capsule of an eye, with the eye focused on an object distant from the viewer;

FIG. 2 is an enlarged fragmentary sectional view of the rearward retinal portion of a human eye depicting the condition of drusen deposit;

FIG. 3 is a front view of the IOL of FIG. 1 with parts broken away to reveal the internal construction thereof, with the IOL in its flattened, rest condition;

FIG. 4 is a vertical sectional view taken along line 4-4 of FIG. 3;

FIG. 5 is a perspective view of the rearward portion of the IOL of FIG. 1;

FIG. 6 is a perspective sectional view of the IOL illlustrated in FIG. 5; and

FIG. 7 is an exploded view of another embodiment of the IOL of the present invention.

### DETAILED DESCRIPTION OF THE PREFERRED EMBODIMENTS

Referring now to the drawings, the present invention is in the form of an telescopic IOL for surgical replacement of the human lens in the treatment of AMD in the human eye. FIG. 1 shows the various components of the human eye 10 pertinent to this invention. Briefly, the eye 10 includes a frontal portion 12 and a rearward portion 14. The frontal portion 12 of the eye 10 is covered by a cornea 16 which encloses and forms an anterior chamber 18. The anterior chamber 18 contains aqueous fluid and is bounded at the rear by an iris 20. The iris 20 opens and closes to admit appropriate quantities of light into the inner portions of the eye 10. The eye 10 also includes a capsule 22 which ordinarily contains the natural crystalline lens (which would be located at numeral 24 in the natural, unmodified eye). The eye 10 includes a ciliary muscle or body 26 having zonular fibers 28 (also referred to as zonules) which are attached to the eye 10.

Ocular adjustments for sharp focusing of objects viewed at different distances is accomplished by the action of the ciliary body 26 on the capsule 22 and natural crystalline lens 24 through the zonular fibers 28. The ciliary body 26 contracts, allowing the capsule 22 to return to a more spherical shape for viewing objects near to the viewer. When the ciliary body 26 retracts, the zonular fibers 28 stretch to make the capsule 22 more discoid thus permitting objects at a distance to be viewed in proper focus. (FIG. 1) To summarize, when the eye 10 focuses, the capsule 22 changes shape to appropriately distribute the light admitted through the cornea 16 and the iris 20. The light then travels through the vitreous humor 30, which is occupied by vitreous fluid, to a retina 32 at the rearward portion 14 of the eye 10. Images received by the retina 32 are transmitted through the optic nerve 34 to the brain.

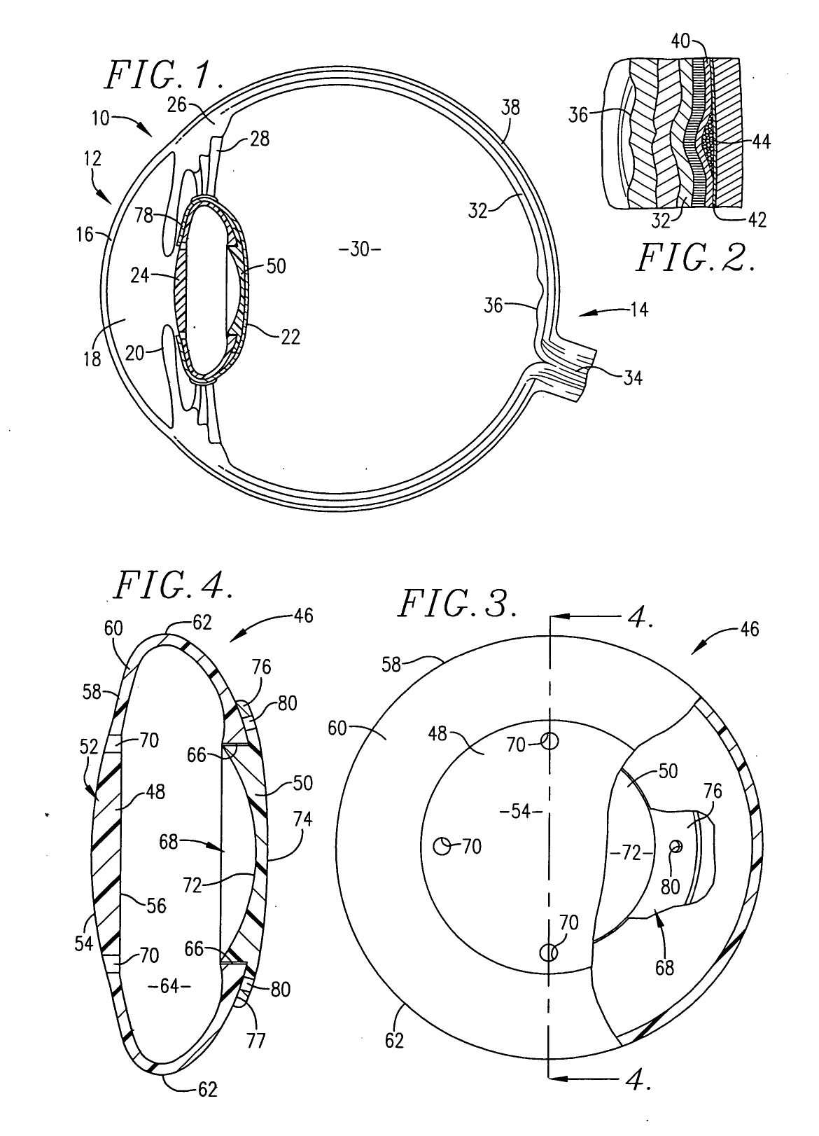

The retina 32 (see FIG. 2) is composed of rods and cones which act as light receptors. The macula 36 is located in the center of the rearward portion of the retina 32 and is responsible for central vision. The outside of the rearward portion 14 of the eye 10 is known as the sclera 38 which joins into and forms a portion of the covering for the optic nerve 34. The layers located beneath the retina 32 include retinal pigmented epithelium (RPE) 40 and Bruch's membrane 42. The RPE 40 is responsible for removing waste products from the retina 32 and preventing new blood vessel growth into the retina 32. Bruch's membrane 42 supports the retina 32. Compromised cellular metabolism of the RPE 40 results in the accumulation of yellowish debris, commonly referred to as drusen deposits 44, between the RPE 40 and Bruch's membrane 42. The accumulation of these drusen deposits 44 results in macular deterioration.

In the natural eye, light passes through the cornea 16 and the iris 20 where the light is refracted onto the natural crystalline lens 24 located in the capsule 22. Light converges at a point directly posterior to the natural crystalline lens 24 where the image is inverted. This inverted image is then brought to focus upon the macula 36 after the light has traveled through the vitreous humor 30. The image is converted to a series of electrical impulses which is then transmitted to the optic nerve 34. The optic nerve 34 carries the information to the brain where the image is translated into its upright position. In a patient with AMD, the degenerate macula 36 prevents proper receipt of the image thereby disabling central focus. Referring to FIGS. 1 and 4-7, an IOL 46 in accordance with the invention comprises an anterior lens member 48 and a posterior lens member 50 operably coupled together by a connector, preferably an annular flange 76 which is arcuate in cross-section, that may change shape in response to zonular movement. The anterior lens member 48 includes an anterior optic 52 presenting an anterior surface 54 and a posterior surface 56. The anterior surface 54 of the anterior optic 52 is convex while the posterior surface 56 of the anterior optic 52 is planar (hereinafter plano-convex). Although the anterior optic 52 is illustrated as plano-convex, any converging optic may be used depending upon the user's eyesight. Examples of converging optic shapes include biconvex and convex meniscus. The anterior optic 52 also comprises four positioning holes 70 located on its periphery that extend therethrough. (See FIG. 4) The four positioning holes 70 are located on the anterior surface 54 of anterior optic 52 at the 12, 3, 6 and 9 o'clock positions. (See FIG. 3) However, the number and location can be varied to meet desired surgical parameters.

The anterior lens member 48 further includes a flexible body 58 integral with the anterior optic 52. Flexible body 58 forms a wall 60. The IOL in cross-section, as shown in FIG. 4, has a wall 60 which in turn extends radially to form opposing bights 62 presenting corresponding termini 66 to mate with posterior lens member 50. Wall 60 is of uniform thickness as it extends posteriorly to meet the posterior lens member 50, until the wall 60 approaches termini 66. The thickness of wall 60 is greater at the termini 66 remote from the anterior optic 52. The ratio in thickness of the termini 66 to the remainder of flexible body 58 is of from about 1:1 to 5:1.

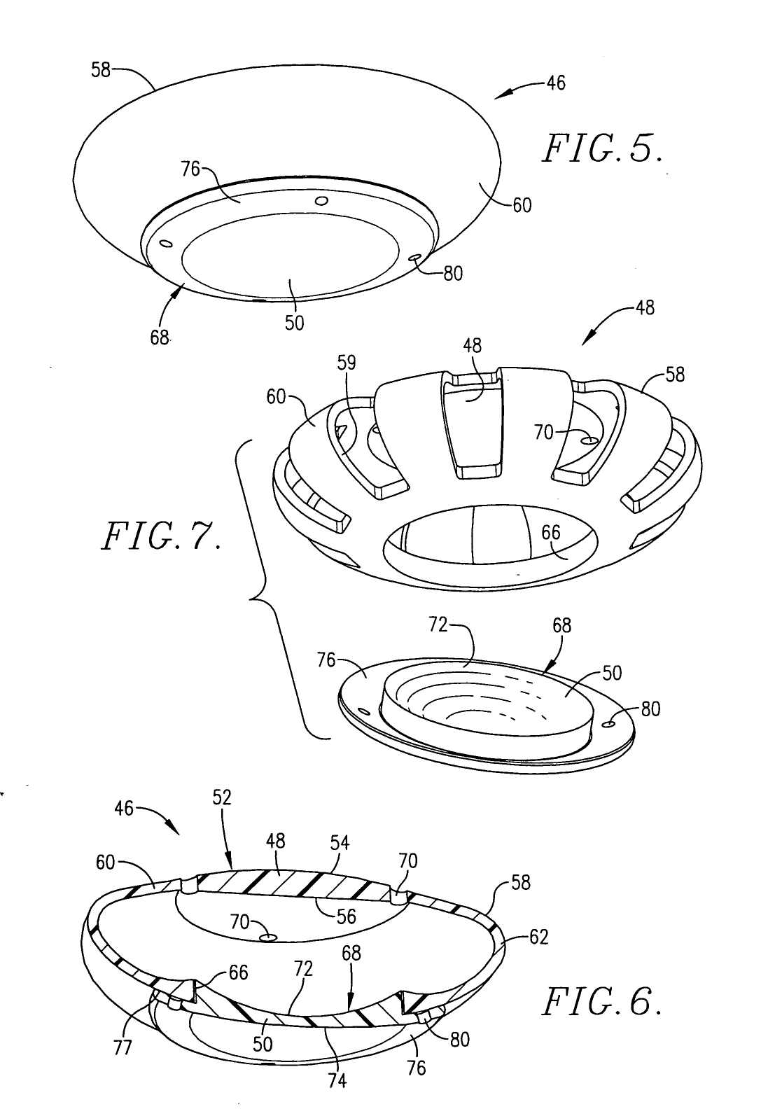

The posterior lens member 50 comprises a posterior optic 68 presenting an anterior surface 72 and a posterior surface 74. The anterior surface 72 of the posterior optic 68 is concave while the posterior surface 74 of the posterior optic 68 is convex (hereinafter concave meniscus). A concave meniscus optic is a diverging optic having a concave anterior surface wherein the concave surface has a lesser radius of curvature than the opposing convex posterior surface. Although the surface of the posterior optic 68 is illustrated as concave meniscus, any diverging optic may be used depending upon the user's eyesight. Examples of diverging optic shapes include biconcave and plano-concave. The posterior optic 68 is integral with an annular flange 76 and mates with termini 66 to form chamber 64. The annular flange 76 surrounds the posterior optic 68 and then extends arcuately from the posterior optic 68 to cover portions 77 of anterior lens member 48. Flange 76 has positioning holes 80 formed therein located at approximately the 3 and 9 o'clock positions. (See FIGS. 3 and 7) However, the number and location of the positioning holes 80 may vary depending upon the desired surgical parameters.

The IOL of the present invention may be formed with arcuate openings 59 formed in the flexible body 58 of the anterior lens member 48 as illustrated in FIG. 7. The embodiment of FIG. 7 can optionally be provided with a very thin membrane (not shown) in covering relationship as disclosed in U.S. patent application Ser. No. 09/940,018, filed Aug. 27, 2001, which is incorporated by reference herein. It is contemplated that the membrane would be formed of the same material as the lens members 48, 50 but would be much thinner (on the order of a few thousandths of an inch) than the remainder of the lens members 48, 50. The purpose of the membrane is to prevent or at least impede the passage of migratory cells through openings within the IOL and into the inner chamber of the IOL.

The IOL of the present invention can be formed of any biologically inert material conventionally used in intraocular lens construction, (e.g., flexible synthetic resin materials). Examples of suitable lens materials include silicones, acrylates (such as polymethylmethacrylates), and mixtures thereof. It is contemplated that mixtures of silicones and acrylates comprise both chemical mixtures, such as silicone-acrylate blends, and various combinations of silicones and acrylates employed to construct the lens. It is particularly preferred that IOLs according to the present invention be constructed of a material having an elastic memory (i.e., the material should be capable of substantially recovering its original size and shape after a deforming force has been removed). An example of a preferred material having elastic memory is MEMORYLENS (available from Mentor Ophthalmics in California).

Preferably the IOL will have an outer equatorial diameter (distance between outer surfaces of opposing bight sections 62) of from about 8 mm to 12 mm. (See FIG. 4) Preferably the IOL will have a polar height (distance between outer surfaces of opposing anterior 58 and posterior 50 lens members) of from about 3 mm to 5.5 mm.

The IOL 46 substitutes for the natural crystalline lens 24 of the human eye 10, and is preferably implanted into the biological capsule 22. In order to insert the inventive lens 46 into the biological capsule 22, an opthalmic surgeon would remove the natural crystalline lens 24 leaving an opening in the capsule 22. The surgeon folds the posterior lens member 50 and inserts it substantially within the capsule 22, using holes 80 therein to position the posterior lens member 50. Because the patient's eye is facing upward during surgery, the folded lens member floats downward into the eye after unfolding itself. After the posterior lens member 50 is inserted, the anterior lens member 48 is also folded and inserted into the biological capsule 22. The anterior lens member 48 also unfolds and fits over the posterior lens member 50 because it substantially fills the capsular bag 22. The surgeon uses the holes 70 on the anterior optic 52 of the anterior lens member 48 to position the anterior lens member 48 into place. The anterior optic 52 and posterior optic 68 are positioned such that both optics share the same optical axis. Surgical implantation of the IOL 46 in this manner is advantageous because it requires a small incision size of from about 1 to 5 mm. At incision sizes of this range, the capsular walls reseal themselves and therefore, do not require sutures which may cause bleeding in the eye.

The instant invention improves vision by magnifying the observed image onto undamaged regions of the retina 32 thereby permitting proper transmittal to the optic nerve 34. Light travels through the cornea 16 and the iris 20 as with a natural eye, however, with the inventive IOL 46, the light is refracted through the plano-convex surface of the anterior optic 52. The inventive IOL replaces the natural crystalline lens 24 of the human eye 10. The anterior optic 52 converges the light. Light is refracted through the posterior optic 68 where it is then magnified and projected onto a large region of the retina 32. The image is brought to focus upon the retina 32 and a series of electrical impulses are transmitted to the brain. The inventive IOL 46 permits an AMD patient to improve vision via utilizing principles of a Galillean telescope to refract light to undamaged portions of the retina 32.

Not only does the IOL of the present invention project an observed image onto undamaged regions of the retina 32, but it also accommodates in response to action of the ciliary body 26 in connection with the zonular fibers 28 to view objects located both near and far from the viewer. When the viewer is observing an image located at a distance, the sensory cells within the retina 32 signal the ciliary body 26 to relax, thus pulling on the zonular fibers 28 to make the capsule 22 more discoid as shown in FIG. 1. In doing so, the polar dimension of the capsule 22 is narrowed, which in turn causes the polar dimension of the IOL 46 to narrow in a similar manner.

The IOL of the present invention typically has a diopter value of from about 16 to 26. The diopter value of a lens is defined as the reciprocal of the focal length in meters:
Diopter=1/focal length (m).
Focal length is the distance from the center of the lens to the object being viewed. The focal length must decrease as magnification increases. The diopter value expresses the refractive capacity of a lens which is associated with the radius of curvature of the optics. Generally, an increased diopter value indicates that the optic is thicker at its center and also has a lesser radius of curvature. Thus, a larger radius of curvature generally permits greater divergence of light.

Although the invention has been described with reference to the preferred embodiment illustrated in the attached drawing figures, it is noted that equivalents may be employed and substitutions made herein without departing from the scope of the invention as recited in the claims. For example, while the foregoing method of inserting the inventive IOL 46 into the capsule 22 presumed that a portion of the anterior wall 78 of the capsule 22 would be removed with the natural crystalline lens 24, it will be appreciated that it may be possible to insert the IOL 46 through an incision in the posterior wall 80 of the capsule 22. While the foregoing description discloses that the IOL 46 could be utilized in AMD patients, the IOL 46 may be used in any situation where the natural crystalline lens 24 needs to be replaced such as with cataracts. Furthermore, the IOL may be utilized in situations when the natural crystalline lens 24 needs to be replaced for both cataracts and AMD.

Having thus described the preferred embodiment of the invention, what is claimed as new and desired to be protected by Letters Patent includes the following:

1. A telescopic intraocular lens for implantation substantially within the capsule of the human eye between the anterior and posterior capsule walls, said lens comprising:
an anterior light-converging optic;
a posterior light-diverging optic; and,
a connector operably connecting said optics, said lens changing shape, when implanted, in response to accommodation of the eye.

2. The lens of claim 1, said lens being formed of a flexible synthetic resin material.

3. The lens of claim 2, said flexible synthetic resin material comprising a biologically inert material.

4. The lens of claim 2, said flexible synthetic resin material comprising a material selected from the group consisting of silicones, acrylates, and mixtures thereof.

5. The lens of claim 1, said lens having a diopter value of from about 16 to 26.

6. The lens of claim 1, said lens having an equatorial diameter of from about 8 mm to 12 mm.

7. The lens of claim 1, said lens having a polar height of from about 3 mm to 5.5 mm.

8. The lens of claim 1, said connector having positioning holes formed therein.

9. The lens of claim 1, said connector further including a flexible body operably coupled to said anterior light-converging optic.

10. The lens of claim 9, said flexible body being integral with said anterior light-converging optic.

11. The lens of claim 9, said flexible body having a pair of opposing bights.

12. The lens of claim 9, said flexible body extending radially outward from said anterior light-converging optic.

13. The lens of claim 9, said flexible body being arcuate in cross-section.

14. The lens of claim 9, said flexible body connected to said posterior light-diverging optic at a connection point, and said flexible body having a first cross-sectional thickness at said connection point and a second cross-sectional thickness at a location other than said connection point, said first cross-sectional thickness being greater than said second cross-sectional thickness.

15. The lens of claim 9, said flexible body connected to said posterior light-diverging optic at a connection point, and said flexible body having a first cross-sectional thickness at said connection point and a second cross-sectional thickness at a location other than said connection point, the ratio of said first cross-sectional thickness to said second cross-sectional thickness being from about 1:1 to 5:1.

16. The lens of claim 1, said connector further including an annular flange operably coupled with said posterior light-diverging optic.

17. The lens of claim 16, said annular flange being arcuate in cross-section.

18. The lens of claim 1, said posterior light-diverging optic having positioning holes formed therein.

19. A telescopic intraocular lens for implantation substantially within the capsule of the human eye between the anterior and posterior capsule walls, said lens comprising:
an anterior lens member including an anterior light-converging optic;
a posterior lens member including a posterior light-diverging optic;
said lens members operably connecting said optics and further being structurally distinct and separable from one another.

20. The lens of claim 19, said lens being formed of a flexible synthetic resin material.

21. The lens of claim 20, said flexible synthetic resin material comprising a biologically inert material.

22. The lens of claim 20, said flexible synthetic resin material comprising a material selected from the group consisting of silicones, acrylates, and mixtures thereof.

23. The lens of claim 19, said lens having a diopter value of from about 16 to 26.

24. The lens of claim 19, said lens having an equatorial diameter of from about 8 mm to 12 mm.

25. The lens of claim 19, said lens having a polar height of from about 3 mm to 5.5 mm

26. The lens of claim 19, said anterior lens member having positioning holes formed therein.

27. The lens of claim 19, said anterior lens member further including a flexible body operably coupled to said anterior light-converging optic.

28. The lens of claim 27, said flexible body being integral with said anterior light-converging optic.

29. The lens of claim 27, said flexible body having a pair of opposing bights.

30. The lens of claim 27, said flexible body extending radially outward from said anterior light-converging optic.

31. The lens of claim 27, said flexible body being arcuate in cross-section.

32. The lens of claim 27, said flexible body connected to said posterior light-diverging optic at a connection point, and said flexible body having a first cross-sectional thickness at said connection point and a second cross-sectional thickness at a location other than said connection point, said first cross-sectional thickness being greater than said second cross-sectional thickness.

33. The lens of claim 27, said flexible body connected to said posterior light-diverging optic at a connection point, and said flexible body having a first cross-sectional thickness at said connection point and a second cross-sectional thickness at a location other than said connection point, the ratio of said first cross-sectional thickness to said second cross-sectional thickness being from about 1:1 to 5:1.

34. The lens of claim 19, said posterior lens member being an annular flange operably coupled with said posterior light-diverging optic.

35. The lens of claim 34, said annular flange being arcuate in cross-section.

36. The lens of claim 19, said posterior lens member having positioning holes formed therein.

37. A method of implanting a telescopic intraocular lens substantially between the anterior and posterior capsule walls of the human eye comprising:
creating a capsule incision of a small size;
positioning a posterior lens member including a posterior light-diverging optic within said capsule adjacent said posterior capsule wall;
positioning an anterior lens member including an anterior light-converging optic within said capsule; and
connecting said lens members.

38. The method of claim 37, further including the step of folding the members.

39. The method of claim 37, said incision size being of from about 1 mm to 5 mm.

40. The method of claim 37, said members sharing substantially the same optical axis.
